(19) United States
(12) Patent Application Publication
(10) Pub. No.: US 2019/0308004 A1
(43) Pub. Date: Oct. 10, 2019

(54) LOW PROFILE SELF-SEALING ACCESS PORT

(71) Applicant: Marius SAINES, Inglewood, CA (US)

(72) Inventor: Marius SAINES, Inglewood, CA (US)

(21) Appl. No.: 16/373,654

(22) Filed: Apr. 3, 2019

Related U.S. Application Data
(60) Provisional application No. 62/652,443, filed on Apr. 4, 2018.

(51) Int. Cl. A61M 39/02; A61M 1/14; A61M 1/36; A61M 39/04

(52) U.S. Cl. CPC A61M 39/0208; A61M 1/14; A61M 2039/0223; A61M 39/04; A61M 1/3661; A61M 1/3655

(57) ABSTRACT

This invention provides for a self-sealing port having a low profile and substantially flat bottom for use in hemodialysis. The self-sealing ports can be repeatedly used for the cannulation required for the blood flows associated with hemodialysis.

The present invention relates to the field of self-sealing ports for repeated cannulation, particularly for use in association with use in dialysis and for AV grafts.

### BACKGROUND OF THE INVENTION

Hemodialysis is now a commonly practiced method of treating patients suffering from renal failure. Hemodialysis machines serve to remove life-threatening chemicals from the blood stream, when the kidneys can no longer effectively remove such chemicals.

In order to perform hemodialysis, access must be obtained to the blood flow system, and blood flows of 150 to 400 ml/minute are required. Blood from veins is inadequate to meet these flow requirements, and repeated puncture of a large artery is not feasible.

The use of AV fistula is considered the best option for hemodialysis. Once an AV fistula is created, it has to become accessible. That process is called maturation. For that two conditions need to be met:

The vein should have a diameter of minimum 6 mm and it should be visible; and If the vein is too deep, or is located in an unfavorable position, as the basilique vein, the solution is a surgical transposition, which is a large operation, requiring long incision. If the diameter of the vein is under 6 mm, it is difficult to stick.

In both this cases, inserting the port will make it accessible for dialysis. Accordingly, a medical procedure has been developed whereby ports are inserted in a line connected to a superficial artery and a nearby vein. See U.S. Pat. Nos. 5,876,366 and 5,876,366. The ports each have a septum.

The ports are implanted entirely below the skin to reduce the risk of infection and to provide better comfort to the patient between dialysis treatments. Placement of the port requires a two inch incision; a hypodermic needle is used to puncture the skin and then the insertion of the port.

The prior art ports have the potential to move, particularly when the septum is being accessed by the hypodermic needle. An additional self-sealing port is shown in U.S. Pat. No. 7,261,705. This port has a number of parts and has a high profile “chimney” 204 with the septum at top, in an excess of 10 mm. Other ports have a depending projection, such as shown in FIG. 2 of U.S. Pat. No. 5,876,366. In U.S. Pat. No. 4,405,319 tissue ingrowth material is used on the port so as to stabilize the port. Such a configuration does not lead to stability of the port and present a large unit to be inserted.

Another application for the port is for AV grafts. When the patients do not have good veins, an artificial graft is inserted between an artery and a vein. After using those grafts, for a year, the wall of the graft gets shredded from multiple punctures, with episodes of bleeding, or aneurysm formation. The grafts need surgical repair with segment replacement. This type of repair can be done by inserting the port, which will stop graft damage

### SUMMARY OF THE INVENTION

The present invention meets the above-described needs by providing a port that is particularly suitable for insertion in a simple medical procedure. The port provides access to a vein and/or artery and is stable, even when the septum is being accessed by a hypodermic needle. The septum is made of a soft silicone material so that after puncture and withdrawal of the needle, the septum seals by itself. This prevents the veins and arteries of the patient from being repeatedly punctured. The material for the soft silicone septum is well known in the art.

The port of the present invention has a low profile for easy insertion of the port in place. The bottom of the port (the side opposite the septum and in contact with the subcutaneous skin) is substantially flat, forming a stable platform for the port even when it is being accessed. The overall thickness of the port of the present invention is between 6 mm and 10 mm, permitting the port to be readily inserted in the body of the user, unlike the prior art ports.

In use, the port is typically held by the nurse or doctor while it is being accessed. While the port of the present invention has application during dialysis, it may also be used in any situation where the patient repeatedly has injections. Further, a large septum area is provided, or multiple septums are provided, permitting the port to be used over a long period of time.

### BRIEF DESCRIPTION OF THE DRAWINGS

FIG. 1 is a top view of the present invention.

FIG. 2 is a side view of the present invention.

FIG. 3 is a left end view along lines 3-3 of FIG. 2 of the present invention.

FIG. 4 is a right end view of the present invention.

FIG. 5 is a right top perspective view of the present invention.

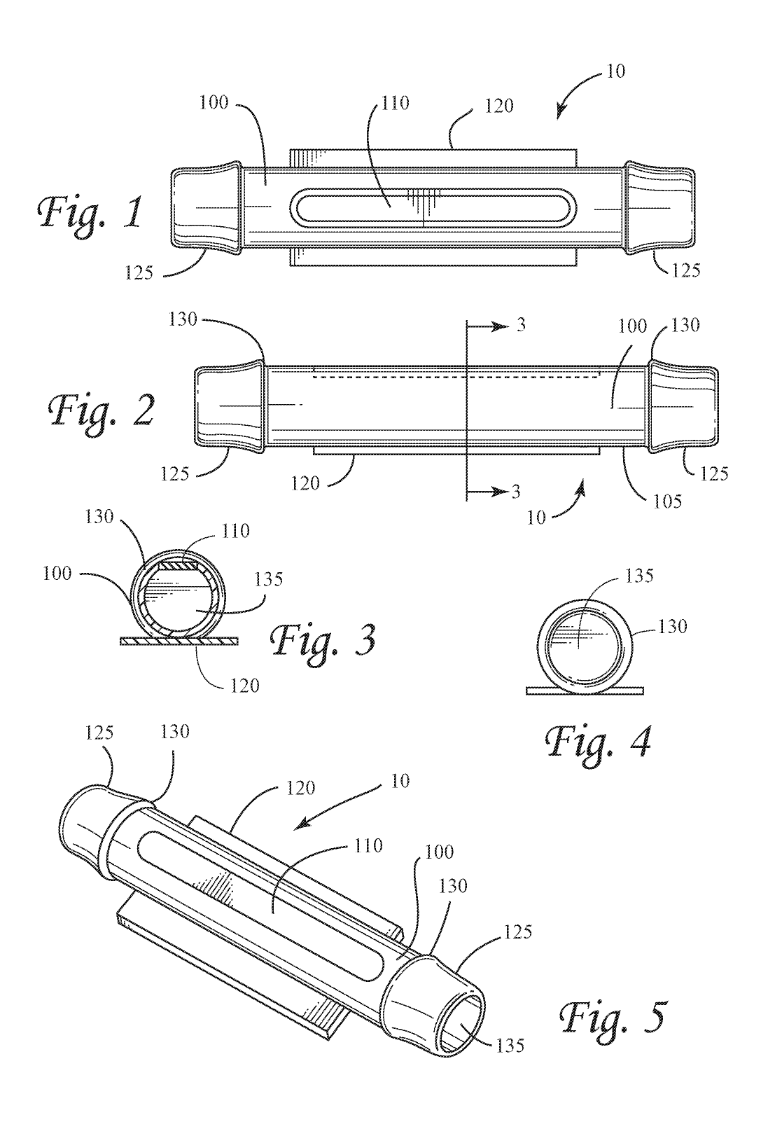

### DETAILED DESCRIPTION OF THE INVENTION

The port 10 of the present invention consists of a hollow tubular member 100 having an approximate overall length of 25 mm. The outer diameter of the hollow tubular member 100 in the preferred embodiment of the present invention is approximately 7 mm and the interior of the hollow tubular member 100 is approximately 6 mm. The total height of the port of the present invention does not exceed 10 mm. A port 10 made of a selastic member 110 is fitted in the top wall of the tubular member 100 and a rectangular support plate 120 is affixed to the bottom of the tubular member 100. The selastic member 110 is of sufficient size and thickness to permit multiple punctures by a needle along the top of this selastic member 110 permitting access by the needle, not shown, into the interior of the tubular member 100. The overall length of the tubular member should preferably be less than 50 mm.

Connection nipples 125 fit over each end of the hollow tubular member 100. The connection nipples 125 have an enlarged portion 130 proximate the hollow tubular member 100. A flexible tube (not shown), such as tetrafluoroethylene, fits over the connection nipples 125 with the other end of the tube connected to the patient's natural vessels. The expanded portion 130 of the connection nipples 125 serves to hold the flexible tubes in place on the port 10. The expanded portion 130 is tapered at its end 34 and 36 to facilitate being attached to the tube 121 and create a leak proof seal.

In the preferred embodiment of the present invention the selastic member 110 of the septum is made of a soft silicone material which can be repeatedly punctured by a needle so that the septum can be used repeatedly. This selastic member 110 covers a substantial portion of the hollow tubular member 100. However a series of individual selastic members can be used instead of a single selastic member. Since the port is placed under the skin of the patient, it is highly desirable that it be able to remain in place as long as possible.

Blood flow is permitted to enter the nipple 125 at one end of the port and pass through the inside 135 of the hollow tubular member 100 and exit from the other connection nipple 125. The port 10 itself is capable of being used in either direction.

In use, the port 10 is surgically inserted beneath the skin of the patient. The flat bottom 120 of the port 10 maintains the port 10 in a stable position so that it is substantially held in place and resists movement when the doctor or nurse is accessing the septum 110 with a needle (not shown).

The port 10 may be made of any suitable plastic material or metal such as titanium. Suitable materials for septum 110 include, but are not limited to, silicon and several other plastic polymers. Various materials for making the septum 110 are well known to those skilled in the art. Preferably, the body of the port is made of a single piece, with the septum inserted in the wall of the hollow tubular member 100 and held in place by any suitable means.

The needle used for puncturing the septum 114 should be a coreless needle, i.e., it should not cut the seal of the septum 114 but should open a hole that may tightly close after the needle withdrawal.

While the invention has been described in connection with certain preferred embodiments, it is not intended to limit the scope of the invention to the particular forms set forth, but, on the contrary, it is intended to cover such alternatives, modifications, and equivalents as may be included within the spirit and scope of the invention as defined by the appended claims.

I claim the following:

1. An implantable blood access port for providing access to the circulatory system comprising a rigid hollow tubular member made of a biologically compatible material, said body being hollow and having an input and an output, said hollow tubular member having a self-sealing septum after puncture by a needle, and a substantially flat member on the bottom of the port.

2. The device of claim 1 in which the thickness of the port is no larger than 10 mm.
